(19) United States
(12) Patent Application Publication
(10) Pub. No.: US 2023/0270783 A1
(43) Pub. Date: Aug. 31, 2023

(54) IMMUNE SYSTEM RESTORATION BY CELL THERAPY

(71) Applicant: THE NATIONAL INSTITUTE FOR BIOTECHNOLOGY IN THE NEGEV LTD., Beer Sheva (IL)

(72) Inventors: Alon Monsonego, Nir-Banim (IL); Yehezqel Elyahu, Yoqne'am Illit (IL)

(21) Appl. No.: 18/043,576

(22) PCT Filed: Aug. 25, 2021
(86) PCT No.: PCT/IL2021/051047

§ 371 Date: Feb. 28, 2023

Related U.S. Application Data
(60) Provisional application No. 63/073,183, filed on Sep. 1, 2020.

(51) Int. Cl. A61K 35/17; C12N 5/0783; A61P 37/02; A61P 25/28; C12Q 1/6886

(52) U.S. Cl. CPC A61K 35/17; C12N 5/0638; A61P 37/02; A61P 25/28; C12Q 1/6886; C12Q 2600/158

(57) ABSTRACT

The present disclosure generally relates to compositions and methods for treating immune system imbalance or for treating immune system associated diseases.

### FIELD OF INVENTION

The present disclosure generally relates to the field of restoring and/or adjusting an immune system of a subject using a cell-based therapy, in particular to immune system restoration and/or adjustment based on subject etiology and/or immune system status evaluation.

### BACKGROUND

A significant change observed in aging relates to the composition and functionally of CD4 T cells, the main orchestrators of adaptive immune responses. With aging, this naïve subset shrinks along with the accumulation of highly differentiated memory cells which often shows dysregulated properties. These changes are assumed to result from age-related thymus involution, repeated antigen encounters and intrinsic cellular senescence processes. In addition, systemic low-grade chronic inflammation that develops with age, also appears to impact the phenotype and function of CD4 T cells.

Unfortunately, the changes in the immune system have a tendency to result in dysregulation and/or impaired function of the immune system, rendering the elderly more prone to so called “inflammaging”—infections, chronic inflammatory disorders, and vaccination failure on the one hand, and to increased occurrence of cancer on the other.

There remains an unmet need to test immune dysfunction in an individual long before or during disease process and to restore and/or adjust the balance of the immune system based on the identified alteration and/or patient etiology.

### SUMMARY

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

According to some embodiments, there is provided compositions and method for treating a dysregulated immune system. According to some embodiments, there is provided compositions and method for treating senescence-associated diseases.

Advantageously, the herein disclosed composition and method enable providing personalized treatment to a subject in need thereof, in particular elderly, according to the status of their immune system, e.g., whether the immune system is identified as balanced, imbalanced making the subject more vulnerable to inflammatory and neurodegenerative diseases or imbalanced making the subject more prone to cancer.

According to some embodiments, there is provided a method or a pharmaceutical composition for use in the treating a senescence-associated disease in a subject in need thereof, the method comprising: obtaining information of a disease etiology of the subject; wherein the disease etiology comprises a senescence-associated disease; and administering to the subject cytotoxic CD4 T-cells (CD4-CTLs) and/or an agent capable of inducing CD4-CTLs differentiation and/or proliferation, thereby treating the senescence-associated disease; or administering an agent capable of aTreg depletion and/or inhibition.

According to some embodiments, the cytotoxic CD4 T-cells are autologous to the subject.

According to some embodiments, the method further comprises a step of isolating effector memory CD4 T-cells (EMs) from the subject and cause their differentiation into CD4-CTLs. According to some embodiments, causing the differentiation comprises cultivating the EMs in the presence of one or more marker selected from IL-27, IL-6, IL1, TNF. According to some embodiments, the isolating of the EMs comprises sorting EMs from the subject using CD44, CD62L, CD45, Itgb7 and/or IL-18R1 as biomarkers. Each possibility is a separate embodiment.

According to some embodiments, the senescence-associated disease is selected from frailty, cancer, chronic infection, chronic inflammation, Alzheimer's disease, dementia, Parkinson's disease, tissue senescence or any combination thereof. Each possibility is a separate embodiment.

According to some embodiments, there is provided a method or a pharmaceutical composition for use in the treating an immune-associated disease of a subject in need thereof, the method comprising: obtaining information of a disease etiology of the subject; wherein the disease etiology comprises immune-inflammatory condition or an immune-insufficient condition; and providing cell-based therapy to the subject based on the disease etiology; wherein the cell therapy comprises administering to the subject aTregs CD4 T cells and/or an agent capable of inducing CD4 aTregs differentiation and/or proliferation or administering to the subject cytotoxic CD4 T-cells (CD4-CTLs) and/or an agent capable of inducing CD4-CTL differentiation and/or proliferation.

According to some embodiments, the cell therapy is autologous cell therapy.

According to some embodiments, the therapy comprises administering to the subject aTregs CD4 T cells and/or an agent capable of inducing CD4 aTregs differentiation. According to some embodiments, the method further comprises a step of isolating and proliferating aTregs CD4 T-cells isolated from the subject. According to some embodiments, the isolating comprises sorting aTregs from the subject using one or more biomarkers selected from CD137, CD134, FOXP3+, GITR+, Helios+, CD74, HLA-DR CD81, TIGIT, PD1. Each possibility is a separate embodiment.

According to some embodiments, the therapy comprises administering to the subject cytotoxic CD4 T-cells (CD4-CTLs) and/or an agent capable of inducing CD4-CTL differentiation and/or proliferation. According to some embodiments, the method further comprises a step of isolating effector memory CD4 T-cells (EMs) from the subject and cause their differentiation into CD4-CTLs. According to some embodiments, causing CD4 CTL differentiation comprises cultivating the EMs in the presence of one or more marker selected from IL-27, IL-6, IL1, TNF. According to some embodiments, the isolating of the EMs comprises sorting EMs from the subject using CD44, CD62L, CD45, Itgb7 and/or IL-18R1 as biomarkers.

According to some embodiments, the immune-inflammatory associated disease etiology is an autoinflammatory and/or autoimmune disease and wherein the cell-based therapy comprises administering to the subject aTregs CD4 T cells and/or an agent capable of inducing CD4 aTregs differentiation and/or proliferation.

According to some embodiments, the immune-insufficient disease etiology is selected from frailty, cancer, chronic infection, chronic inflammation, Alzheimer's disease, dementia, Parkinson's disease, tissue senescence. Each possibility is a separate embodiment. According to some embodiments, the cell-based therapy comprises administering to the subject cytotoxic CD4 T-cells (CD4-CTLs) and/or an agent capable of inducing CD4-CTLs differentiation and/or proliferation.

According to some embodiments, there is provided a method or a pharmaceutical composition for use in the restoring and/or adjusting an immune system of a subject, the method comprising: obtaining a biological sample from a subject, the biological sample comprising one or more subsets of CD4 T-cells; identifying the presence, frequency and/or ratio of cytotoxic CD4 T-cells (CD4-CTLs), exhausted CD4 T-cells and/or aTreg cells, and providing cell-based therapy to the subject based on the identification, thereby restoring and/or adjusting the immune system of the subject.

According to some embodiments, the identifying of the presence of one or more subsets of CD4 T-cells comprises determining the amount of each identified subset of CD4 T-cells relative to a control value.

According to some embodiments, the method further comprises identifying the presence, frequency and/or ratio of one or more additional subsets of CD4 T cells in the immune system, selected from activated regulatory CD4 T cells (aTregs), effector memory CD4 T-cells (EMs); naïve CD4 T-cells, naïve_Isg15 CD4 T-cells, rTregs CD4 T-cells or any combination thereof. Each possibility is a separate embodiment.

According to some embodiments, the evaluating is based on the level of one or more biomarkers associated with the CD4-CTLs. According to some embodiments, the biomarker is EOMES.

According to some embodiments, the evaluating is based on a plurality of biomarkers selected from Nkg7, Runx3, EOMES, Gzmk, IFN-b, IFN-g, IL-27, IL21, IL 17A, Ccl3, Ccl4 and Ccl5. Each possibility is a separate embodiment.

According to some embodiments, the therapy comprises administering to the subject CD4 aTregs and/or an agent capable of inducing CD4 aTregs differentiation and/or proliferation. According to some embodiments, the aTregs CD4 T cells are autologous to the subject. According to some embodiments, the method further comprises a step of isolating and optionally also proliferating aTregs CD4 T cells of the subject. According to some embodiments, the isolating comprises sorting aTregs from the subject using one or more biomarkers selected from CD137, CD134, FOXP3+, GITR+, Helios+, CD74, HLA-DR, CD81, TIGIT, PDJ. Each possibility is a separate embodiment.

According to some embodiments, the therapy comprises administering to the subject an agent targeting the CD4-CTLs. According to some embodiments, the agent is selected from the group consisting of an antibody, a siRNA, a microRNA, a small molecule or any combination thereof. Each possibility is a separate embodiment. According to some embodiments, the antibody is an NKG2D antibody, a CD7 antibody, a CD134 antibody, a CD137 antibody, a GITR antibody, a CCL5 antibody, an IL-27 antibody or any combination thereof. Each possibility is a separate embodiment.

According to some embodiments, the therapy comprises administering to the subject CD4-CTLs and/or an agent capable of inducing CD4-CTL differentiation and/or proliferation. According to some embodiments, the CD4-CTLs are autologous to the subject. According to some embodiments, the method further comprises a step of isolating effector memory CD4 T-cells (EMs) from the subject and cause their differentiation into CD4-CTLs. According to some embodiments, the isolating of the EMs comprises sorting EMs from the subject using CD44, CD62L, CD45, Itgb7 and/or IL-18R1 as biomarkers. Each possibility is a separate embodiment.

According to some embodiments, the method further comprises evaluating a grade of tissue senescence, based on the presence, frequency and/or ratio of cytotoxic CD4 T-cells (CD4-CTLs), exhausted CD4 T-cells, aTreg cells or combinations thereof.

According to some embodiments, there is provided a method or a pharmaceutical composition for use in the evaluating tissue senescence in a subject in need thereof, the method comprising evaluating obtaining data relating to the subjects age, medical history and/or genetic background, measuring the presence, frequency and/or ratio of cytotoxic CD4 T-cells (CD4-CTLs), exhausted CD4 T-cells, aTreg cells or combinations thereof, and assessing the degree of tissue senescence in the subject based on the data relating to the subjects age, medical history and/or genetic background and the identified presence, frequency and/or ratio of cytotoxic CD4 T-cells (CD4-CTLs), exhausted CD4 T-cells, aTreg cells or combinations thereof.

According to some embodiments, the data obtained from the subject include at least the subject's age and his/her medical history.

According to some embodiments, at least the presence, frequency and/or ratio of cytotoxic CD4 T-cells (CD4-CTLs) is measured.

According to some embodiments, the method further comprises determining a likely location of the senescent tissue based on the medical history of the subject.

According to some embodiments, there is provided a method or a pharmaceutical composition for use in the treatment of cancer of a subject, the method comprising administering to the subject CD4-CTLs and/or an agent capable of inducing CD4-CTL differentiation and/or proliferation.

According to some embodiments, the CD4-CTLs are autologous to the subject. According to some embodiments, the method further comprises a step of isolating effector memory CD4 T-cells (EMs) from the subject and cause their differentiation into CD4-CTLs. According to some embodiments, the isolating of the EMs comprises sorting EMs from the subject using CD44, CD62L, CD45, Itgb7 and/or IL-18R1 as biomarkers. Each possibility is a separate embodiment.

According to some embodiments, there is provided a pharmaceutical composition for treating an immune system imbalance, the composition comprising isolated CD4 T-cells and one or more excipients.

According to some embodiments, immune system imbalance is related to a senescence-associated disease and the CD4 T-cells are CD4 CTLs. According to some embodiments, the senescence-associated disease is selected from frailty, cancer, chronic infection, chronic inflammation, Alzheimer's disease, dementia, Parkinson's disease, tissue senescence or any combination thereof.

According to some embodiments, the immune system imbalance is related to an autoinflammatory and/or autoimmune disease and the CD4 T-cells are CD4 aTregs.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures.

FIG. 1A shows pie charts presenting the percentage of cells belonging to each of the seven subsets in a young mouse and an old mouse.

FIG. 1B. is a scheme illustrating the major changes that occur in the population of CD4 T cells during aging.

FIG. 2A shows representative flow cytometry plots (left) and corresponding analysis (right) presenting the prevalence of exhausted cells defined as PD1⁺CD62L⁻ cells (pink/positive slope; R=0.94) and naïve cells, defined as CD62L⁺PD1⁻ out of CD4⁺EOMES⁻CCL5⁻FOX3⁻ cells (blue/negative slop; R=0.96) measured via flow cytometry in mice at the age of 2 and 16 months, where shaded areas of each graph (right) represent ±s.e.m.

FIG. 2B shows representative flow cytometry plots (left) and corresponding analysis (right) presenting the prevalence of aTregs cells defined as CD81⁺CD44⁺ cells out of CD4⁺FOXP3⁺CD8⁻ cells (R=0.78) measured via flow cytometry in mice at the age of 2 and 16 months, where shaded areas the graph represents ±sem.

FIG. 2C shows representative flow cytometry plots (left) and corresponding analysis (right) presenting the prevalence of cytotoxic cells defined as EOMES⁺CCL5⁺ out of CD4⁺CD8− cells (R=0.91) measured via flow cytometry in mice at the age of 2 and 24 months, where shaded areas the graph represents ±sem.

FIG. 3A shows representative flow cytometry plots (left panel) of sorted CD25^{high}CD81⁻ (yellow/rTregs) and CD25^{high}CD81⁺ (brown/aTregs) and corresponding bar plot (right panel) presenting the suppression ability (%) of rTregs versus aTregs (each dot represents a mouse (n=8, from two independent experiments; unpaired T test (*p<0.05).

FIG. 3B shows percentage of cells positive to pro-inflammatory and cytotoxic cytokines (IFNγ, Granzyme B, TNF and Perforin) in Effector Memory (EM) cells, exhausted CD4 T cells and CD4 cytotoxic (CTL) cells 48 hours after activation with anti-CD3/anti-CD28 beads (Dynabeads™, Gibco), measured by flow cytometry. Lines connect measurements within the same mouse. Data from two independent experiments, n=7 mice. Paired T test (*p<0.05, **p<0.01, ***p<10-3, ****p<10-4).

FIG. 4A shows the relative frequency of total CD4 T cells in peripheral blood mononuclear cell (PBMC) obtained from young and old healthy human subjects.

FIG. 4B shows the relative frequency of naïve CD4 T cells (Sell) in peripheral blood mononuclear cell (PBMC) obtained from young and old healthy human subjects.

FIG. 4C shows the relative frequency of CD4 cytotoxic T cells (CD4-CTLs) in peripheral blood mononuclear cell (PBMC) obtained from young and old healthy human subjects.

FIG. 5 shows EOMES^{lox/lox} PCR validation using primers recommended by Jackson (stock number 017293) FIG. 6A shows mean and standard deviations (SDs) of wheel activity (n=6-8) in old mice (each dot represents one mouse).

FIG. 6B shows mean and SDs of grip test (Y axis=s×g) and hanging test (Y axis=Newtons).

FIG. 6C shows mean and SDs of food intake, water intake (n=6-8) in old mice (each dot represents one mouse).

FIG. 6D is a heat-map showing correlations between the frequency of CD4 T cell subsets (naïve, exhausted, memory, and CD4-CTLs) and the physical and metabolic tests (n=6-12). All correlations were calculated assuming the data exhibit a Gaussian distribution (Pearson correlation).

FIG. 7A shows the experimental outline for evaluating the correlations of between CD4 T-cell subsets and biomarkers of aging. In short, old mice (18-24 months) undergo a physical and metabolic assessment using the metabolic cages. Mice were monitored for 48 h using the PROMETHION system (Sable systems, NV). Subsequently, they were killed and analyzed for inflammatory cytokines and chemokines in serum samples using Multiplex ELISA, IHC analysis for senescent cell in liver tissues, and CD4 T-cell subsets analysis using flow cytometry

FIG. 7B shows a heat-map of the correlation between the frequency of CD4 T cell subsets (naïve, exhausted, memory, CD4-CTL's) and CD8 cells, with physical and metabolic tests (food and water intake in g, wheel activity and overall activity in m/48 h and energy expenditure (EE) in Kcal/hr (n=12)). All correlations were calculated assuming the data exhibit a Gaussian distribution (Pearson correlation).

FIG. 7C shows bubble chart presents the correlations between wheel activity(m), wheel speed (m/s), overall activity (m) and percentage of cytotoxic CD4 T cells. each dot represents one mouse.

FIG. 7D shows a chart presenting the activity(m) and wheel activity(m) during day and night cycles. The red line represents the mice with high CD4-CTL levels while the blue line represents mice with low CD4-CTL levels.

FIG. 7E shows a heat-map of the correlation between cytokines and physical/metabolic tests (n=12). All correlations were calculated assuming the data exhibit a Gaussian distribution (Pearson correlation).

FIG. 7F shows IHC staining for p16 and p21 (senescence markers) around the central vein in liver tissue from old mice with high CD4-CTL levels (right image) and old mice with low CD4-CTL levels (left image). Cell nuclei (white) are marked with DAPI.

FIG. 8A shows the experimental outline for evaluating the effect of injecting young spleenocytes into the spleens of old mice splenocytes from young (1-month-old) CD45.1 mice were injected into two groups: young B6 WT mice (2 months old) and old B6 WT mice (26 months old). Thirty days after the CD45.1 cell injection the spleens were analyzed via flow cytometry.

FIG. 8B shows histograms of flow cytometry analysis performed according to the experimental outline of FIG. 8A. CD4 T cells were gated as CD45.1+CD3+CD4+, CD4 Treg cells as CD4+ FOXP3+, effector memory cells as CD44+CD62L− and naïve CD4 T cells as CD62L+CD44−.

FIG. 8C shows representative flow cytometry plots of the gating strategy of cytotoxic CD4 T cells.

FIG. 8D shows histograms of flow cytometry analysis performed according to the experimental outline of FIG. 8A. cytotoxic CD4 T cells were gated as CD45.1+CD3+CD4+EOMES+CCL5+, exhausted CD4 T-cells were gated as CD45.1+CD3+CD4+CD44+PD1+.

FIG. 8E shows the percentage of CD4-CTLs (left) and exhausted effector cells (CD44+PD1⁺) in the endogenous CD4 T cells population (CD45.2) as compared to the exogenous CD4 T cells (cd45.1) population. Paired T test.

FIG. 9A shows the experimental outline for evaluation T-cell subsets in a mouse model of liver fibrosis and senescence (carbon tetrachloride treated mice CCL4 as compared to PBS injected).

FIG. 9B shows representative images of a histological assessment ((hematoxylin and eosin staining in upper panels and Sirus Red staining in lower panels) of livers harvested according to the experimental outline of FIG. 9A (p-value 0.0001>).

FIG. 9C shows histograms of flow cytometry analysis performed according to the experimental outline of FIG. 9A. The liver CD4-CTLs were gated as EOMES+CCL5+ out of CD4+CD8− cells (P value=0.0355).

FIG. 9D shows histograms of flow cytometry analysis performed according to the experimental outline of FIG. 9A. The levels of exhausted cells (gated as PD1⁺ LAG3+), Treg cells (gated as FOXP3+) and effector memory CD4 cells (CD44+CD62L−) were evaluated in carbon tetrachloride treated (CCL4) versus mock-treated (PBS) mice.

FIG. 10A shows the experimental outline for evaluation T-cell subsets in carbon tetrachloride treated (CCL4) mice in a Eomes KO mouse model (CreER+/−) as compared to WT (CreER−/−).

FIG. 10B shows histograms gating CD4-CTLs (EOMES+CCL5+) out of the total CD4+CD8− cells in response to CCl4 injections in Eomes KO mice and WT mice (p value=0.0473).

FIG. 10C shows representative images of a histological assessment ((hematoxylin and eosin staining in upper panels and Sirus Red staining in lower panels) of livers harvested according to the experimental outline of FIG. 10A (P-value 0.0001>).

FIG. 10D shows histograms of flow cytometry analysis (percentage-left and intensity (MF)-right). The level of exhausted cells (gated as PD1⁺ LAG3+) in KO mice (CreER+/−Eomes+/+) in response to CCL 4 injection was evaluated and compared to that of WT mice (CreER −/−), (p-value=0.0482).

FIG. 10E shows histograms of flow cytometry analysis. The level of Treg cells (gated as FOXP3+) in KO mice (CreER+/−Eomes+/+) in response to CCL 4 injection was evaluated and compared to that of CCL4 treated WT mice (CreER −/−), (p-value=0.0482).

FIG. 11A shows the tumor volume mm³ over time after injection of high-grade tumor cells into young mice (black line) or young mice (green/grey line).

FIG. 11B shows the tumor volume mm³ over time after injection of low-grade tumor cells into young mice (black line) or young mice (green/grey line).

FIG. 12A is a violin plot of the percentage of CD4 T-cells out of the total CD3-positive population within the tumor harvested from mice injected with high-grade (blue/dark grey) or low-grade (pink/light grey).

FIG. 12B is a violin plot of the percentage of CD4 T-cells out of the total CD3-positive population in spleens harvested from mice injected with high-grade (blue/dark grey) or low-grade (pink/light grey) or control (orange/hourglass shape).

FIG. 13A is a violin plot of the percentage of CD4 CTLs out of the total CD4 T-cell population within the tumor harvested from mice injected with high-grade (blue/dark grey) or low-grade (pink/light grey).

FIG. 13B is a violin plot of the percentage of CD4 CTLs out of the total CD4 T-cell population in spleens harvested from mice injected with high-grade (blue/dark grey) or low-grade (pink/light grey) or control (orange/hourglass shape).

FIG. 13C is a violin plot of the percentage of naïve CD4 T-cells out of the total CD4 T-cell population within the tumor harvested from mice injected with high-grade (blue/dark grey) or low-grade (pink/light grey).

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The complexity of the immune system, and, particularly, the large variety of the cells comprising it, renders its investigation challenging. Until recently, the various cell lineages of the immune system were explored and traced either by using known cell markers or by analyzing bulk populations; however, these analyses cannot detect small populations of cells or novel subtypes of cells whose markers are yet unknown. These difficulties may be addressed by the single-cell RNA-sequencing (scRNA-seq) technology—a technology that provides RNA-sequencing profiles for hundreds and even thousands of single cells, which are then characterized and clustered in an unbiased manner. Each cluster can be associated with a potentially new marker gene, and the population structure can be assessed at a larger scale. For example, applying this technology, lymphopoiesis was shown to be decreased with age and CD4 T cells were shown to demonstrate a higher cell-to-cell variability in the expression of core activation programs in older ages.

As used herein, the term “biomarker” refers to a nucleic acid sequence of a gene or a fragment thereof the expression of which is indicative of one or more subsets of CD4 T cells. The biomarker may be a serum biomarker released into circulation. Alternatively, the biomarker may be expressed at the cell surface of CD4 T cell. The biomarker may be DNA, mRNA or the cDNA corresponding thereto, which represent the gene or a fragment thereof. A polynucleotide may comprise modified nucleotides, such as methylated nucleotides and nucleotide analogs. The sequence of the biomarker may be interrupted by non-nucleotide components. A biomarker may be further modified after polymerization, such as by conjugation with a labeling component. The term also includes both double- and single-stranded molecules.

As used herein, the term “biomarker associated with the one or more subsets of CD4 T cells” may refers to any measurable indicator of the one or more subsets of CD4 T cells, such as expression levels (including single cell expression levels) of RNA and/or proteins associated with certain CD4 T cell phonotypes, such as but not limited to, CD4 cytotoxic cells and/or activated regulatory (aTreg) CD4 T cells. According to some embodiments, the markers (or some thereof) may be indicative of the subset of CD4 T-cells regardless of the activation status (whether activated or not). Alternatively, the markers (or some thereof) may be indicative of the subset of CD4 T-cells and their activation status (e.g. activated CD4 cytotoxic cell).

As used herein, the term “biomarker identifier” may refer to any molecule capable of identifying a biomarker. Non-limiting examples of biomarker identifiers include, RNA/DNA probes, primers, antibodies etc.

As used herein the term “CD4 cytotoxic cell” refers to a subset of CD4⁺ T cells with cytotoxic activity (CD4-CTL). These cells are characterized by their ability to secrete granzyme B and perforin and to kill the target cells in an MHC class II-restricted fashion.

As used herein the term “regulatory CD4 T cells” or “rTreg” refer to a subpopulation of CD4+ T cells that modulate the immune system, maintain tolerance to self-antigens, and prevent autoimmune disease. As used herein the term “activated regulatory CD4 T cells” or “aTreg” refer to a subpopulation of Treg cells with an activated phenotype and a very strong inhibitory function on T cell proliferation.

As used herein, the term “exhausted CD4 T cells” refer to a subpopulation of CD4⁺ T cells characterized by poor effector functions and high expression of multiple inhibitory receptors.

As used herein the term “effector-memory T cells” or “TEM” refer to a subpopulation of antigen-experienced and long-surviving cells CD4+ T cells characterized by distinct homing capacity and effector function.

As used herein, the term “cell-based therapy” may refer to a therapy configured to boost, activate, inhibit, enlarge the population of or otherwise change the functionality and/or activity and/or distribution of a particular CD4 cell population. According to some embodiments, the cell-based therapy may include administration cells of a CD4 cell subset (also referred to herein as “cell-therapy”. As a non-limiting example, the cell therapy may include administration of CD4-CTLs or of aTreg. According to some embodiments, the cell-based therapy may include administration of an agent capable of inhibiting a particular CD4 cell subset. As a non-limiting example, the agent may be an siRNA targeting Eomes, thereby inhibiting CD4-CTLs. As another non-limiting example, the agent may be an antibody (e.g. an NKG2D antibody). According to some embodiments, the cell-based therapy may include administration of an agent capable of inducing/inhibiting proliferation and/or differentiation of a CD4 T-cell subset, such as but not limited to IL-27, IL-6, IL1, TNF or combinations thereof.

As used herein, the term “biological sample” may refer a sample obtained from a subject which is a body fluid or excretion sample including, but not limited to, seminal plasma, blood, peripheral blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, peritoneal fluid, cyst fluid, lavage of body cavities, broncho alveolar lavage, lavage of the reproductive system and/or lavage of any other organ of the body or system in the body and stool. Each possibility is a separate embodiment of the present invention.

In some embodiments, the biological sample, also termed hereinafter ‘the sample’, obtained from the subject comprises blood. In some embodiments, the sample obtained from the subject is peripheral blood. In some embodiments, the sample obtained from the subject comprises serum. In some embodiments, the sample obtained from the subject is a sample of serum.

In some embodiments, the term “peripheral blood”, as used herein, refers to blood comprising of red blood cells, white blood cells and platelets. Typically, the sample is a pool of circulating blood. According to some embodiments, the sample is a peripheral blood sample not sequestered within the lymphatic system, spleen, liver, or bone marrow.

In some embodiments, the sample is a plasma sample. In some embodiments, the sample is a plasma sample derived from peripheral blood.

As used herein, the term “isolate” of a biological sample refer to a subset, derivative or extract derived from the sample. A non-limiting example of an isolate of a biological sample are white blood cells derived from a blood sample. Another non-limiting example includes a T-cell population or a CD4 T-cell population derived from a blood sample.

As used herein the term “functionality” when referring to CD4 T-cells, e.g., CD4 cytotoxic T-cells or Treg cells refers to the “behavior” of the cells after their activation. According to some embodiments, the functionality of the CD4 T-cells may refer to the profile and/or level of cytokines and/or chemokines secreted by the cells (e.g., anti-CD3/anti-CD28, PMA, ConA). According to some embodiments, the profile and/or level of cytokines and/or chemokines secreted may provide an additional layer of validation regarding the status of the immune system (e.g., that the cells are dysregulated).

The term “control value” and “predetermined threshold” (with referral to presence, frequency and/or ratio of CD4 T cells or CD4 T-cell subset), as used herein refers to a standard or reference value which represents the average, standard or normal number of CD4 T cells. This value can be a single value obtained from a single measurement or a mean value obtain from multiple measurements and/or multiple CD4 cell populations and/or CD4 cell populations derived from multiple biological samples. In some embodiments, the control value is a mean value obtained from a plurality of biological sample derived from human subjects. In some embodiments, the control value is an age-matched control. In some embodiments, the terms ‘control value’ and ‘age-matched control” are exchangeable. In some embodiments, the control value comprises young threshold value, also termed hereinafter regulated, efficient and/or naïve threshold value and old threshold value also termed hereinafter dysregulated, aged, mature and/or exhausted threshold value, the former is calculated from a plurality of biological sample derived from young human subjects and the latter is calculated from a plurality of biological sample derived from old human subjects.

In some embodiments, the predetermined threshold with regards to presence, frequency and/or ratio of cytotoxic CD4 T-cells (CD4-CTLs) is about 1%, about 2%, about 5%, about 10%, about 20%, about 30% or about 40% of the total CD4 T-cell population. Each possibility is a separate embodiment.

In some embodiments, when the presence of CD4-CTLs is above about 2%, or above about 5%, above about 10%, above about 20%, above about 30% or above about 40% of the total CD4 T-cell population, the immune system is evaluated as being pro-inflammatory, as tissue undergoing senescence. Each possibility is a separate embodiment.

In some embodiments, when the presence of CD4-CTLs is below 0.5%, below about 1%, below about 2%, below about 5%, below about 10% or below about 20% of the total CD4 T-cell population, the immune system is evaluated as being immune-insufficient. Each possibility is a separate embodiment.

According to some embodiments, when tissue is evaluated as undergoing senescence and/or when a senolytic treatment is desired, the therapy may include administering CD4-CTLs and/or an agent capable of inducing differentiation and/or proliferation of CD4-CTLs. According to some embodiments, evaluation of senescence comprises evaluating the level of one or more senescence markers (e.g., p21 and/or p16) and/or the level of CD4-CTLs and/or identifying low grade systemic inflammation.

According to some embodiments, there is provided a method for evaluating tissue senescence grade, based on the level of CD4-CT1s measured in blood and/or in the tissue. According to some embodiments, the evaluation further comprises taking into account the age of the subject. According to some embodiments, the evaluation further comprises taking into account the medical history and/or genetic background of the subject.

According to some embodiments, when the level of CD-4 CTLs increases above 1%, 2%, 5%, 8% or 10% of the total CD4 T-cell population, the subject has tissue senescence. Each possibility is a separate embodiment. According to some embodiments, the region of tissue senescence may be estimated based on the subject's medical history and/or genetic background.

According to some embodiments, the CT4 T-cells and/or the agent capable of inducing differentiation and/or proliferation of CD4-CTLs is administered systemically, e.g., by IV-injection. According to some embodiments, the CT4 T-cells and/or the agent capable of inducing differentiation and/or proliferation of CD4-CTLs is administered locally e.g., by injection into senescent tissue.

According to some embodiments, the threshold with regards to presence, frequency and/or ratio of cytotoxic CD4 T-cells (CD4-CTLs) for evaluating the immune system as proinflammatory may be the same as the threshold with regards to presence, frequency and/or ratio of cytotoxic CD4 T-cells (CD4-CTLs) for evaluating the immune system as immune-insufficient. For example, the immune system may be evaluated as proinflammatory if the presence, frequency and/or ratio of CD4-CTLs is above 10% and as immune-insufficient if below 10% of the total CD4 T-cell population.

According to some embodiments, the threshold with regards to presence, frequency and/or ratio of cytotoxic CD4 T-cells (CD4-CTLs) for evaluating the immune system as proinflammatory may be different than the threshold with regards to presence, frequency and/or ratio of cytotoxic CD4 T-cells (CD4-CTLs) for evaluating the immune system as immune-insufficient. For example, the immune system may be evaluated as proinflammatory if the presence, frequency and/or ratio of CD4-CTLs is above 20% and as immune-insufficient if below 10% of the total CD4 T-cell population.

According to some embodiments, the threshold may be age related. As a non-limiting example, the threshold with regards to presence, frequency and/or ratio of cytotoxic CD4 T-cells (CD4-CTLs) for evaluating the immune system as proinflammatory may be lower in young adults as compared to elders.

In some embodiments, the term “a plurality”, as used herein, refers to at least two. According to some embodiments, the term “a plurality” refers to at least 2, 3, 4, 5, 6, 7, 8, 9, 10, 11, 12, 13, 14, 15, 16 and 17. Each possibility is a separate embodiment.

In some embodiments, “detecting a level of a biomarker” comprises assessing the presence, absence, quantity or relative amount (which can be an “effective amount”) of each biomarker in the plurality of biomarkers, within a clinical or subject-derived sample, including qualitative or quantitative concentration levels of such biomarker.

In some embodiments, “detecting a level of a biomarker” comprises determining the expression level of each biomarker of said plurality of biomarkers or determining the amount, or relative amount, of DNA or cDNA corresponding to the expression level of mRNA biomarker(s).

In some embodiments, the plurality of biomarkers are selected from the group consisting of: EOMES, CCL3, CCL4, CCL5, CCR7, CD7, CD8, CD74, CD137, CD134, CD25, CD44, CD62L, CD81, CD200, Cst7, Ms4a4b, NKG2D, Nfatc1, Runx2, Runx3, Tbx21, GzmB, GzmK, perforin, FOXP3, GITR, Helios, Lgals1, IGFbp4, LAG3, IL-1a, IL-1b, IL1R2, IL2RA, IL-6, IL-10, IL-17A, IL-21, IL-18R1, IL-27, IFN-b, IFN-g, Isg15, PD1, Lef1, Lfit3, MCP1, Satb1, Ccr7, Aw112010, S100a10, S100a11, S100a4, Sell, Pdcd1, Izumo1r, Ikzf2, Igfbp4, Itgb1, Itgb7, GM-CSF, Sostdc1, Tbcld4, TNF, TNFRSF4, TNFSF8, TNFRSF8, Ct1a2a, Ct1a4 and TNF-RSF9/4. Each possibility is a separate embodiment.

In some embodiments, the plurality of biomarkers comprises at least two biomarkers. In some embodiments, the plurality of biomarkers comprises at least three biomarkers. In some embodiments, the plurality of biomarkers comprises at least four biomarkers.

According to some embodiments, obtaining a biological sample comprising tissue or fluid is carried out by any one or more of the following collection methods blood sampling, urine sampling, stool sampling, sputum sampling, aspiration of pleural or peritoneal fluids, fine needle biopsy, needle biopsy, core needle biopsy and surgical biopsy, and lavage. Each possibility is a separate embodiment of the present invention. Regardless of the procedure employed, once a biopsy/sample is obtained the level of the plurality of biomarkers can be determined and evaluation can thus be made.

The proportion and identity of a pharmaceutically acceptable excipients used in the pharmaceutical composition may be determined by the chosen route of administration, compatibility with live cells, and standard pharmaceutical practice. Generally, a pharmaceutical composition is formulated with components that do not destroy or significantly impair the biological properties of the active ingredients.

In some embodiments, the pharmaceutical composition is administered locally, e.g., in a tumor, or systemically.

The term “treating” as used herein refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of spread or development of the disease or condition, delay or slowing of disease progression, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently.

As used herein, the term “inflammaging” refers to age associated infections, chronic inflammatory disorders, such as but not limited to arthritis.

In some embodiments, the subject in need thereof is a subject in need of treatment or prevention, is human. In some embodiments, the human subject is over the age of 60, over the age of 70 or over the age of 80. In some embodiments, the human subject is is suffering from a immune associated disorder (optionally regardless of age).

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The terms “subject”, “patient” or “individual” generally refer to a human, although the methods of the invention are not necessarily limited to humans and should be useful in other mammals.

### Examples

Materials and Methods

Mice

WT C57BL/6 and CD45.1 (B6.SJL-Ptprca Pepcb/BoyJ) mice were purchased from the Jackson Laboratory (Bar Harbor, Me.) and were housed under specific pathogen-free conditions at the animal facility of Ben-Gurion University. WT C57BL/6 Mice were kept in different age batches from 2 to 24 months. All mice were checked for any macroscopic abnormalities (according to the Jackson guide—“AGED C57BL/6J MICE FOR RESEARCH STUDIES”). Animals with skin lesions, organ specific problems, or behavioral issues were discarded from the study. All surgical and experimental procedures were approved by the Institutional Animal Care and Use committee (IACUC) of Ben-Gurion University of the Negev, Israel.

Tissue Processing for Flow Cytometry and In Vitro Assays

Spleen: see Materials and methods, section “Samples processing for single-cell RNA sequencing (scRNA-seq)”.

Lymph nodes: Mice were killed with overdose of isoflurane and lymph nodes were harvested from inguinal, mesenteric, cervical and axillar areas. Then, lymph nodes were mashed into 70 m cell strainer and cells were washed and counted.

Blood: Blood was collected into EDTA-coated tubes (MiniCollect, Greiner Bio-One) from euthanized mice using cardiac puncture. Red blood cells were then lysed using blood lysis buffer (BD bioscience) and the remaining leukocytes were washed twice and counted.

Bone marrow: Mice were killed with overdose of isoflurane. Femurs and tibias were collected. Cells from the bone marrow were obtained by flushing the bones with injected sterile PBS. Red blood cells were removed using 500 μl ACK lysis buffer for 1.5 minutes.

Flow Cytometry

For extracellular staining, cells were washed with FACS staining buffer (PBS supplemented with 2% FBS and 1 mM EDTA) and incubated with Fc receptor blocker (TrueStain fcX; BioLegend) for 5 minutes at 4° c. To differentiate between live and dead cells, a viability staining step was done using an eFluor780-Fixable Viability dye (eBioscience) following manufacture instructions. Cells were then incubated with primary antibodies for 25 minutes at 4° c. and were washed twice with a FACS staining buffer. The following antibodies were used for membranal staining: PE-conjugated anti-CTLA4 (4C10-4B9; BioLegend), PE/cy7-conjugated anti-CD25 (3C7; BioLegend), AF700-conjugated anti-CD62L (Mel-14; BioLegend), BV605 or BV785-conjugated anti-PD1 (29F.1A12; BioLegend), APC-conjugated anti-CD81 (Eat-2; BioLegend), FITC-conjugated anti-CD8 (53-6.7; BioLegend), PerCP/cy5.5-conjugated anti-CD44 (IM7; BioLegend), AF700 or BV785-conjugated anti-CD4 (RM4-5; BioLegend), PE-conjugated anti-CD121b (4E2; BD Biosciences), BV421-conjugated anti-CD25 (PC61; BioLegend), BV605-conjugated anti-CD195 (C34-3448; BD Biosciences), BV785-conjugated anti-LAG3 (C9B7W; BioLegend), BV421-conjugated anti-CD4 (GK1.5; BioLegend), PerCP/cy5.5-conjugated anti-CD8 (53-6.7; BioLegend), PE-conjugated anti-CD137 (17B5; BioLegend), PE/cy7-conjugated anti-CD134 (OX-86; BioLegend) and PE-conjugated anti-CD178 (MFL3; eBioscience). After staining for membranal markers, intracellular labeling was performed: Cells were fixed and permeabilized using the FOXP3/Transcription factor staining kit (eBioscience), blocked with Rat serum (1 μl per 100p of staining buffer) and stained with the following antibodies: BV605-conjugated anti-TNF (MP6-XT22; BioLegend), BV605-conjugated anti-IL17a (TC11-18H10.1; BioLegend), FITC or BV510-conjugated anti-IL2 (JES6-5H4; BioLegend), BV421 or BV786-conjugated anti-IFN-γ (XMG1.2; BioLegend), BV421-conjugated anti-IL10 (JES5-16E3; BioLegend), APC-conjugated anti-Granzyme B (QA16A02; BioLegend), PE-conjugated anti-CCL5 (2E9/CCL5; BioLegend), PE/cy7-conjugated anti-EOMES (Dan11mag; eBioscience), AF488-conjugated anti-FOXP3 (150D; BioLegend), APC-conjugated anti-IL21 (#149204; R&D systems), PE/dazzle-conjugated anti-Helios 22F6; Biolegend) and APC-conjugated anti-Perforin (B-D48; BioLegend). All flow cytometry experiments were performed with the CytoFLEX instrument (Beckman Coulter). Data were analyzed with the FlowJo (v-10.5.3) software. Gating strategies were set based on fluorescence minus one (FMO), unstained samples and unstimulated samples (when needed). All the samples in the experiment excluded dead cells, clumps and debris.

Clustering Analysis of Flow Cytometry Data

Clustering analysis of flow cytometry data was done using FlowJo (v-10.5.3). First dead cells, doublets and non-lymphocyte cells were excluded (based on viability staining and FSC/SSC channels). CD4⁺ cells were used for further analysis. Data were sampled using “down sampling” function to get 40,000 representative cells from each sample. Then, a t-SNE algorithm was applied with the following parameters: Iteration=1000, Perplexity=40, Learning rate (eta)=2800. Mean fluorescence intensity (MFI) projected on the t-SNE plots for each protein to infer the cluster identity.

Suppression Assay

For in vitro suppression assay, naïve CD4⁺ T cells were isolated from spleens of young (2 months) CD45.1 mice using naïve isolation kit (EasySep™ Mouse Naïve CD4⁺ T Cell Isolation Kit, StemCell Technologies), labelled with CFSE (CellTrace™ proliferation kit, Invitrogen) and used as responder cells (2×104 cells per well). Then, cells were cultured in 96-well plates with irradiated 2×104 APCs (as feeder cells) in the presence of sorted CD25^{high}CD81⁻ or CD25^{high}CD81⁺ Tregs at 1:1, 1:2 and 1:4 responders:Tregs ratios. Cells were stimulated with anti-CD3 (1 μg/ml) for 72 hours. Proliferation (defined as all cells with CFSE dilution) of responder cells was analyzed to assess the suppression of Tregs cells. The percentage of suppression was determined as (100—(% of proliferating cells with Tregs)/(% of proliferating cells without Tregs)).

Serum Cytokines Measurement

Mouse peripheral blood was extracted after right atrial puncture into a 2 ml Eppendorf. Then, blood tubes were incubated at room temperature for coagulation (15 minutes). After incubation, tubes underwent centrifugation step (450 g), and serum was collected. For cytokines measurement, LEGENDplex mouse inflammation kit (BioLegend) was used following manufacture instruction. Data were acquired on CytoFLEX instrument (Beckman Coulter), and analyzed using LEGENDplex analysis software.

Statistical Analysis for Flow Cytometry Experiments

Spearman correlation between the age of mice and the proportions of RECs and naïve cells in spleen was computed in R v3.4.2 using stats package v3.4.1. For statistical analysis GraphPad Prism (version 7.0a) was used. Paired T test was used for comparisons between two groups from the same biological samples. For analysis of more than two group, one-way ANOVA was used and corrected by Bonferroni correction for multiple comparisons.

### Example 1: CD4 T Cells Undergo Extensive Diversification with Age, Resulting in a New Population Structure

In order to classify CD4 T-cell subsets in an unbiased manner, cells were clustered by their transcriptomic profiles and the robustness of the clusters' identity assessed. Seven distinct and robust clusters were identified.

Of the seven distinct clusters, four were matching established subsets: two populations of naïve T cells overexpressing Sell, Lef1 and Igfbp4 genes, which differ by the expression of Isg15 gene (denoted naïve and naïve_Isg15); a population of resting regulatory T cells (rTregs), labeled based on their classical expression of Foxp3 and II2ra genes, together with the expression of naïve-associated genes Lef1 and Sell; and effector-memory T cells (TEM) expressing the S100a4, Igals1 and Itgb1 genes. The transcriptional signatures of the three remaining subsets have not been previously defined in the context of aging, and include: activated regulatory T cells (aTregs) overexpressing Foxp3, Cd81, Cd74 and Cst7 genes, together with aTregs-associated genes such as Tnfrsf4, Tnfrsf9, Tnfrsf18 and Ikzf2 genes; cells with an exhaustion signature (denoted exhausted) overexpressing the Lag3, Tbcld4, Sostdc1 and Tnfsf8 genes; and cells overexpressing genes such as EOMES, Gzmk and Ct1a2a, which are commonly associated with CD8 T cells (denoted cytotoxic), and were previously described in the context of viral infection and cancer as CD4 cytotoxic T cells.

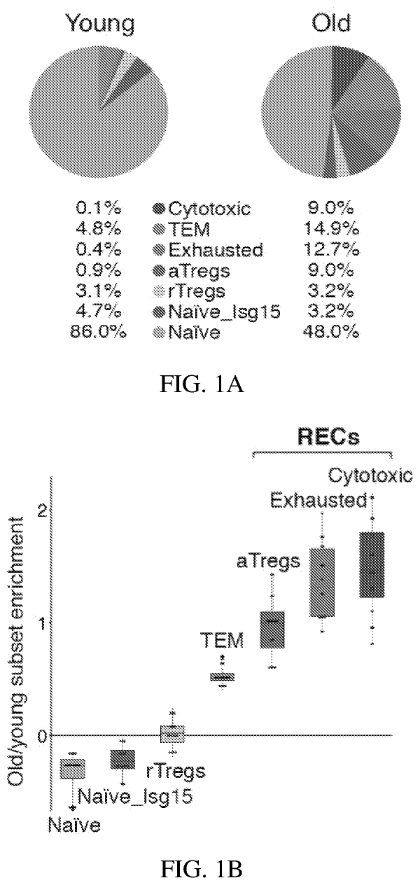

Next, the proportion of each subset in old versus young mice was compared and is presented in Table 1, below and further illustrated in FIG. 1B.

TABLE 1
Proportions of cellular subsets in a young mouse and an old mouse.

| | Presence in young | Presence in old |
|---|---|---|
| Cell subset | mice (%) | mice (%) |
| Cytotoxic | 0.1 | 9 |
| TEM | 4.8 | 14.9 |
| Exhausted | 0.4 | 12.7 |
| aTregs | 0.9 | 9 |
| rTregs | 3.1 | 3.2 |
| Naïve_Isg15 | 4.7 | 3.2 |
| Naïve | 86 | 48 |

As seen, naïve subsets were enriched in young mice (Naïve: log(median)=−0.27, p=0.03, and Naïve_Isg15: log(median)=−0.23, p=0.03). rTregs subset was equally distributed (log(median)=0.02, p=0.89). Four subsets were enriched in every old mouse: TEM (log(median)=0.51, p=0.03), aTregs (log(median)=1, p=0.03), exhausted (log(median)=1.32, p=0.03) and cytotoxic (log(median)=1.46, p=0.03) subsets. Whereas the two naïve subsets were significantly enriched in young mice, the rTregs subset had a similar abundance in both age groups, while the TEM subset was dominant in old mice. Notably, the aTregs, exhausted, and cytotoxic subsets (collectively denoted RECs to represent these Regulatory, Exhausted and Cytotoxic subsets) were highly enriched in all aged mice, accounting for ˜30% of the CD4 T cells and were negligible in young mice (˜1%).

Overall, the results demonstrate that aging is marked by a complex landscape of CD4 T cells, with expansion of subsets with effector (including TEM, exhausted and cytotoxic cells) and regulatory (aTregs) signatures, associated with serum markers of chronic inflammation.

### Example 2: RECs are Distinct CD4 T-Cell Subsets that Gradually Accumulate with Age

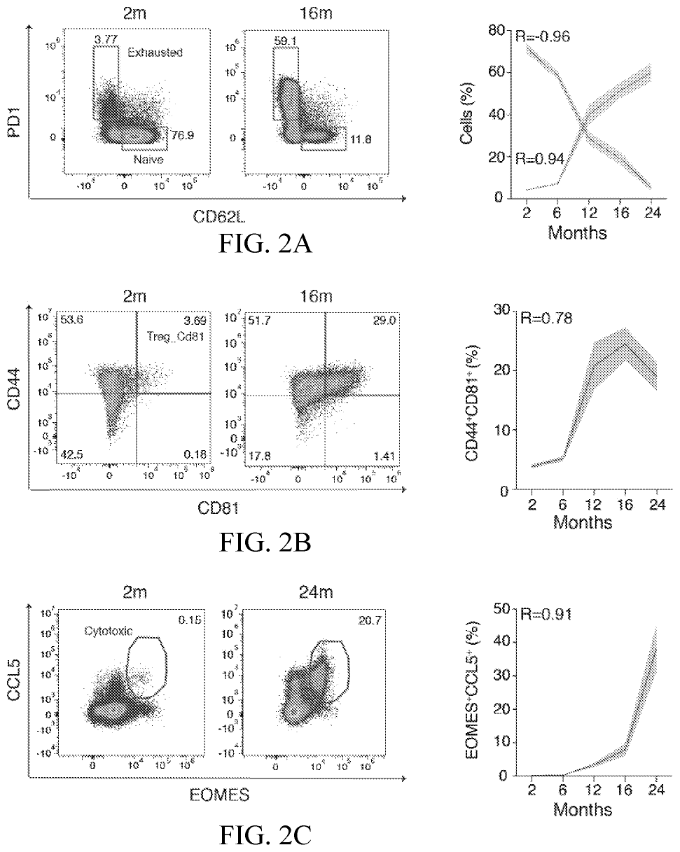

To assess the dynamics of RECs over time, their relative abundance in spleens of healthy mice at 2, 6, 12, 16 and 24 months of age, was measured using flow cytometry. Exhausted cells (defined as CD4+PD1+CD62L-FOXP3-EOMES-CCL5-, steadily accumulated from 6 months of age (r=0.94, p=1.5 ×10-12, Spearman correlation), and coincided with continuous decreased proportions of naïve cells (defined as CD4+CD62L+PD1-CD81−EOMES-CCL5— r=−0.96, p=1.7×10-14, Spearman correlation; FIG. 2A). Out of the regulatory cells (CD4⁺FOXP3⁺), the relative abundance of aTregs, (defined as CD81⁺CD44⁺) also increased with age, reaching a peak at 16 months of age and slightly declining at 24 months (r=0.78, p=10-8, Spearman correlation; FIG. 2B). Cytotoxic cells (defined as CD4⁺EOMES⁺CCL5⁺CD8−), were observed at a later time point at 12 months, and their fraction sharply increased with age (r=0.91, p=2.2×10-16, Spearman correlation; FIG. 2C). Gating in this analysis was based on unstained samples and fluorescence minus one (FMO). Data include three (FIG. 2B and FIG. 2C) or two (FIG. 2A) different experiments, and n=5-10 per time point.

### Example 3: RECs Exhibit Extreme Regulatory and Effector Properties

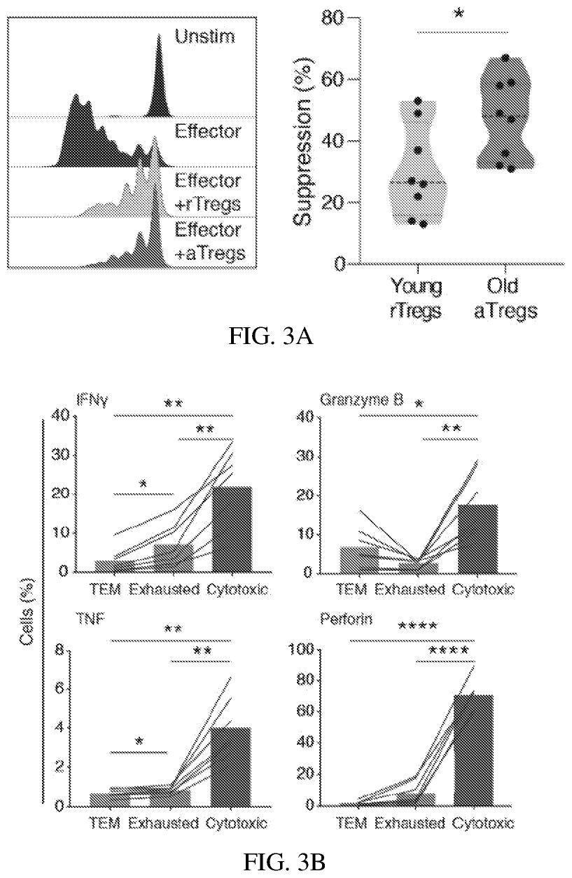

To assess suppressive function of Tregs aTregs (CD25highCD81+; FIG. 3A: brown) from old mice (16 months) were sorted and their suppressive function was compared to that of rTregs (CD25highCD81−; FIG. 3A: yellow) isolated from young (2 months) mice. Suppressive function was assessed ex-vivo after 72 hours of co-culture with activated naïve CD4 T cells from young CD45.1 mice. The reduction in the proliferation of the activated CD4 T cells was measured via flow cytometry and calculated as % of suppression. Surprisingly, aTregs exhibited significantly higher suppressive activity than rTregs ex-vivo (FIG. 3A).

### Example 4: Clinical Studies—Healthy Elderly Individuals Accumulate Cytotoxic CD4 T Cells

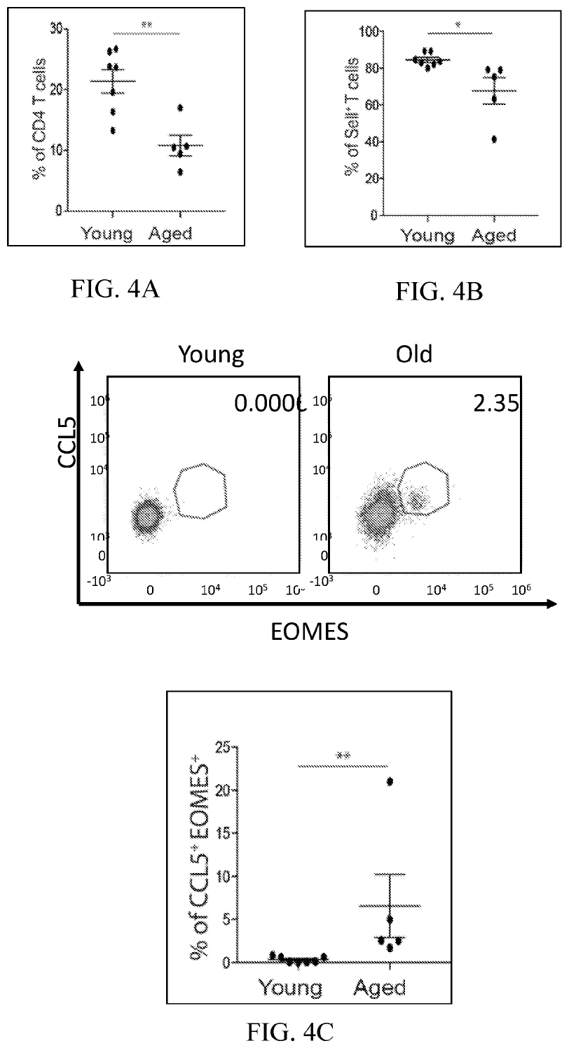

A preliminary clinical study in human subjects revealed that CD4-CTLs accumulate also in elderly, but not in adult, healthy human individuals, as shown in FIG. 4A-FIG. 4C and detailed below.

Blood PBMCs was obtained from healthy young (age: 25-35 years, n=7) and old (age: >70 years, n=5) human individuals and analyzed by flow cytometry. As shown in FIGS. 4A and 4B, the frequency of total CD4 T (FIG. 4A) and naïve CD4 T (FIG. 4B) cells was decreased in old compared with young PBMCs.

In addition, the frequency of CD4-CTLs is higher in elderly than in young human individuals, where in young individual these cells are barely detected (FIG. 4C, t-test, *p<0.05, **p<0.01).

### Example 5: Sorting and Differentiation of Effector Memory (EM) Cells into CD4-CTLs

EM cells were live sorted by FACS using CD4+CD62L-CD44+CCL5+PD1low as markers. The sorted cells are subsequently expanded in the presence of circulating inflammatory cytokines such as but not limited to IL-27, IL-6, IL1, TNF etc. and evaluated for cytotoxic activity by FACS for the presence of EOMES, GrzK IFNg and/or other CD4-CTL markers.

### Example 6: Generation of CD4CreER—EOMES^{lox/lox} Mice

The re-organization of the CD4 T-cell compartment with aging—and, specifically, at the stage where the CD4-CTL subset sharply increases to 30-40% of the CD4 T-cell compartment-may provide protection from tumors and chronic viral infections; however, it can also facilitate chronic inflammation, declined immunity, and killing functions, which can result in significant tissue damage and severe defects in overall immunity and tissue repair.

Since EOMES is expressed in CD4-CTLs and is essential for their inflammatory and cytotoxic function, mice incapable of producing CD4-CTLs were generated.

To this end, CD4CreER mice were crossed with EOMES^{lox/lox} mice and administered IP with TMX at 12, 13, and 14 months of age, i.e., when the CD4-CTLs accumulate.

CD4-CreER, EOMES^{lox/lox}, ROSA^{mT/mG}, OTII⁺ TCR and C57BL/6 CD45.1⁺ mice were purchased from the Jackson Laboratory (Bar Harbor, Me.) and housed under specific pathogen-free conditions at the animal facility of Ben-Gurion University of the Negev, Israel (BGU). Mice are kept in different age batches, from 2 to 24 months old, and routinely monitored for pathogens and health issues. All surgical and experimental procedures will be approved by the Institutional Animal Care and Use committee (IACUC) of BGU.

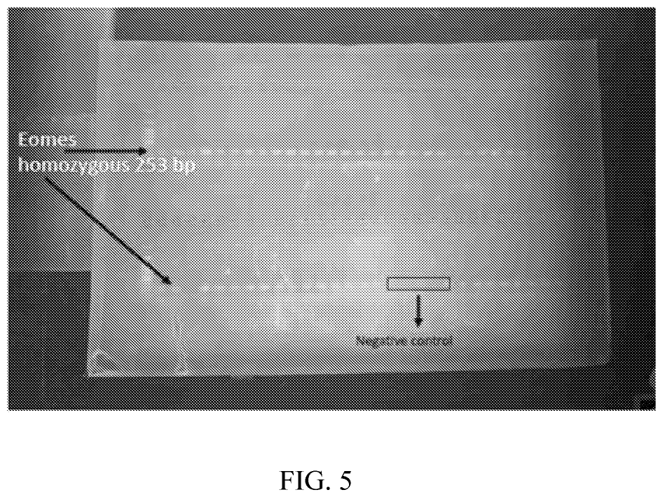

EOMES^{lox/lox} genotype was confirmed by PCR as shown in FIG. 5.

### Example 7: Exploring the Impact of the Cytotoxic CD4 T-Cell Subset on Immune Decline and Chronic Inflammation in Mice

In order to evaluate the impact of CD4-CTLs on chronic inflammation, CD4^{CreER}-EOMES^{lox/lox} mice are administered IP with TMX at 12, 13, and 14 months of age, i.e., when the CD4-CTLs usually accumulate. Two weeks after the final TMX administration, the mice are analyzed for aging biomarkers and subsequently injected with the influenza vaccine or with an adjuvant alone and analyzed, 14 d later, for presence of naïve, CM, EM, exhausted, Treg, and CD4-CTL subset composition in the blood, BM, and spleen, as compared with littermate controls. Splenocytes are stimulated with an influenza lysate and response of the CD4 T-cell subset is analyzed with ELISA and flow cytometry.

Serum samples are analyzed for influenza-specific antibodies and for an array of cytokines and chemokines, including, but not limited to, IL-27, GM-CSF, IL-1b, IL-6, TNF-a, IFNb, IFN-g, IL-17A and CCL2.

### Example 8: Exploring the Impact of the Cytotoxic CD4 T-Cell Subset on Aging Associated Phenotypes

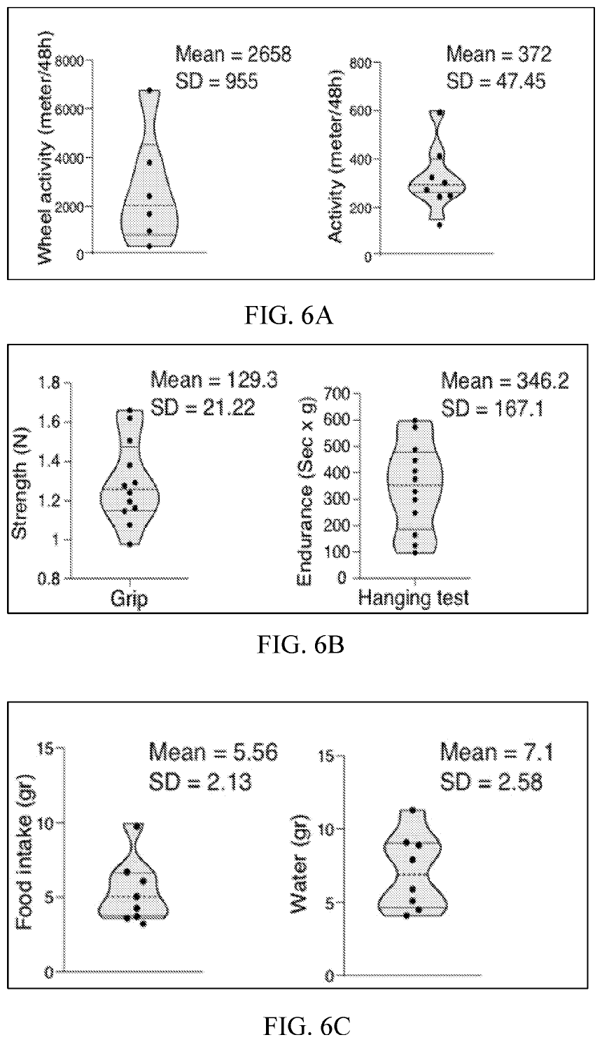

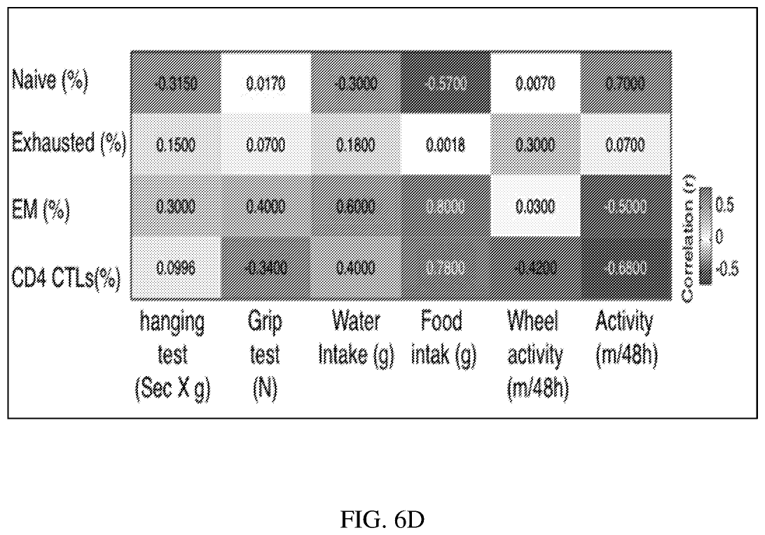

Rotarod test: CD4^{CreER}-EOMES^{lox/lox} mice and littermate controls are trained on the RotaRod for 3 days at speeds of 4, 6, and 8 rounds per minute (RPM) for 200 seconds. On the test day, mice will be placed onto the RotaRod, starting at 4 RPM and accelerating to 40 RPM over 5 min trials. The speed is recorded when the mouse drops off the RotaRod. Results are averaged from 3 or 4 trials and normalized to the baseline speed of young mice. The data is compared to those obtained for wt mice, as shown in FIG. 6A.

Hanging Test: For the hanging test CD4^{CreER}-EOMES^{lox/lox} mice and littermate controls are placed onto a 2-mm-thick metal wire placed 35 cm above a padded surface. The mice are allowed to grab the wire with their forelimbs only. Hanging time is normalized to body weight as hanging duration (sec)×body weight (g). Results are averaged from 2-3 trials for each mouse. The data is compared to those obtained for wt mice, as shown in FIG. 6B.

Grip Test: Forelimb grip strength is performed using a Grip Strength Meter (Columbus Instruments, Columbus, Ohio) for CD4^{CreER}-EOMES^{lox/lox} mice and littermate controls. Results are averaged over 3 or 4 trails. The data is compared to those obtained for wt mice, as shown in FIG. 6B.

Metabolic Tests: A comprehensive metabolic and physical monitoring is performed using the PROMETHION system (Sable systems, NV, USA). Daily activity, wheel usage, sleeping, food intake, water intake and gas exchange will be recorded over a 48h period. The data is extracted using expeData software (Sable systems, NV, USA), and analyzed using prism 8.2.1 (GraphPad). The data is compared to those obtained for wt mice, as shown in FIG. 6C.

The correlation between the frequency of CD4 T cell subsets (naïve, exhausted, memory, and CD4-CTLs) and the physical and metabolic phenotypes of CD4^{CreER}-EOMES^{lox/lox} mice is further compared to the correlation observed for wt mice (FIG. 6D) in order to further assess the effect of CD4-CTLs on the overall distribution of CD4 T-cells.

### Example 9: Exploring the Impact of CD4-CTL Depletion on Aging Associated Phenotypes

Wildtype C57BL6 mice aged 18-20 months are treated IP once a week with anti-IL27 (25 microgram/mouse). After 4 injections, the mice undergo analysis for frailty and metabolic parameters (as described in FIG. 6A-FIG. 6D). Subsequently, the mice are sacrificed and analyzed for age-related CD4 subsets (CD4-CTLs, exhausted, EM, aTregs), and for levels of inflammation and senescence markers, such as, but not limited to, IL-27, GM-CSF, IL-1b, IL-6, TNF-a, IFNb, IFN-g, IL-17A and CCL2 in liver, lung and brain.

### Example 10—CD4 CTL-Cell Subsets and Biomarkers of Aging

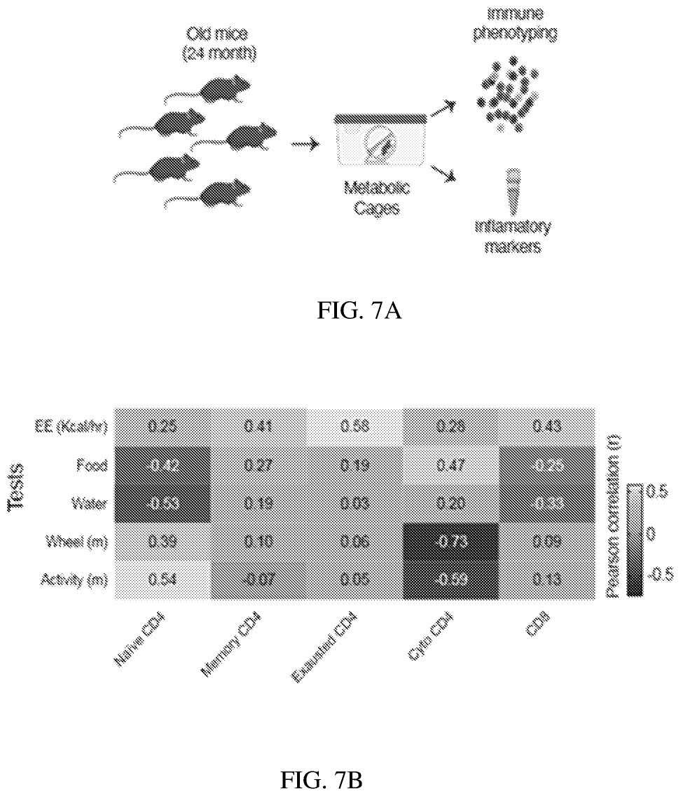

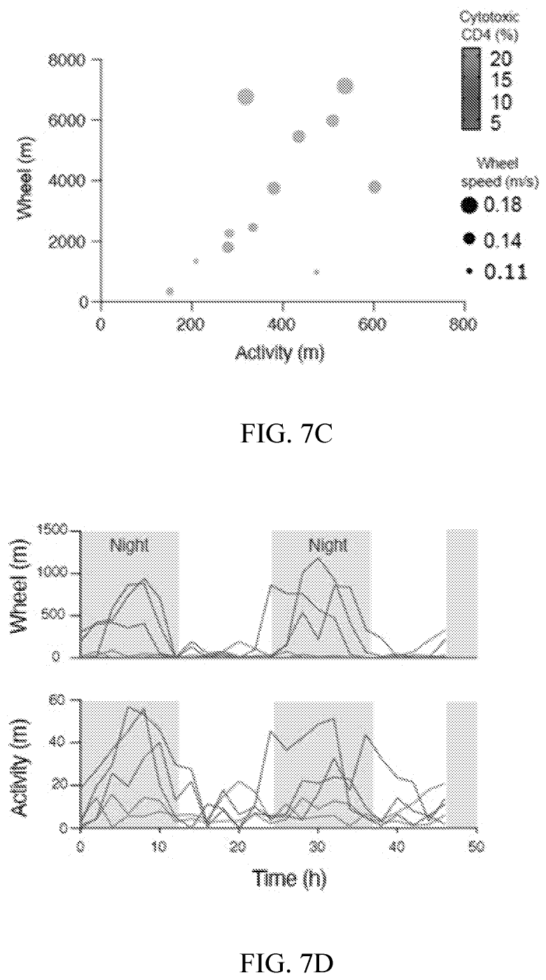

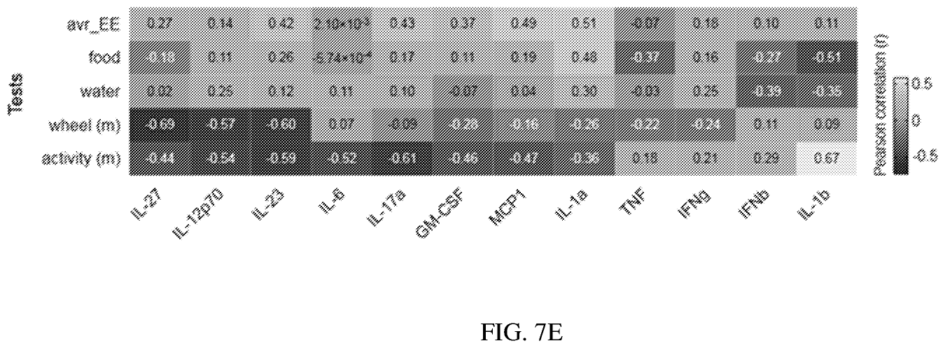

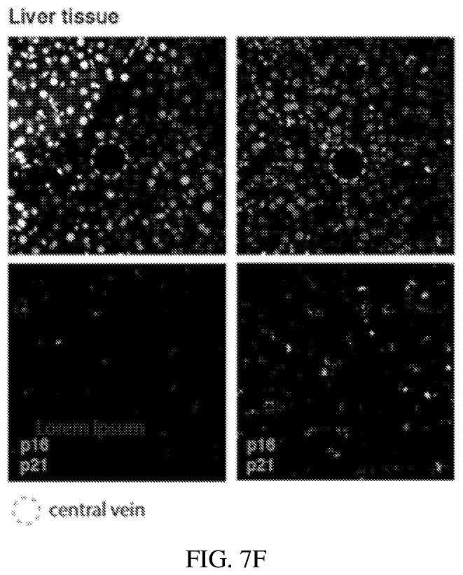

In order to evaluate the correlation between CD4-CTLs and ageing, old mice (18-24 months) underwent a physical and metabolic assessment using metabolic cages. The mice were monitored for 48 h using the PROMETHION system (Sable systems, NV). Subsequently, the mice were sacrificed and analyzed for their level of inflammatory cytokines and chemokines in serum samples using Multiplex ELISA, Immunohistochemistry (IHC) analysis for senescent cell in liver tissues, and CD4 T-cell subsets analysis using flow cytometry (see FIG. 7A).

A heat-map showing correlations between the frequency of CD4 T cell subsets (naïve, exhausted, memory, CD4-CTL's) and CD8 cells, and the physical and metabolic tests (including food and water intake in g, wheel activity overall activity in m/48h and energy expenditure (EE) in Kcal/hr (n=12)) was generated. All correlations were calculated assuming the data exhibit a Gaussian distribution (Pearson correlation). As seen from FIG. 7B, a significant negative correlation was found between the activity of the mice and the abundance of CD4-CTLs.

Moreover, when a per mice correlation between was calculated between wheel activity (m), wheels speed (m/s), overall activity (m) and the percentage of CD4-CTLs a high variability between mice was observed, but a tight correlation between low activity and high percentage of CD4-CTLs was observed (red dots in FIG. 7C).

As mice are known to be particularly active during the night the correlation was between CD4-CTL level and the activity(m) and wheel activity(m) was further evaluated while splitting the activity into day and night cycles. As seen from FIG. 7D, mice with low cytotoxic CD4-CTL levels (blue lines) showed high night activity, while no such boost in activity during night tome was observed in mice with high cytotoxic CD4-CTL levels (red lines).

In order to evaluate the correlation between ageing and certain easily detectable biomarkers, a heat-map was generated, assuming the data exhibit a Gaussian distribution (Pearson correlation). As seen from FIG. 7E, a significant negative correlation between activity and cytokines associated with cytotoxic CD4 cells (CD4-CTLs) was observed, whereas a significant positive correlation was found between activity and IL-1b levels (marker of inflammation).

A similar correlation was found between abundance of p16 and p21 (senescence markers) around the central vein in liver tissue and high CD4-CTL levels, as seen from the representative images of immunohistochemistry stainings (FIG. 7F) for p16 (red) and p21 (green) in a mouse with high CD4 CTL levels (right panel) and a mouse with low CD4-CTL levels (left panel). Cell nuclei (white) were marked with DAPI. These indicate ta role of CD4-CTLs in inhibition of cell senescence.

### Example 11—Spleenocytes from Young CD45.1 Mice, Stimulated by an Old Environment, Undergo Cytotoxic Changes

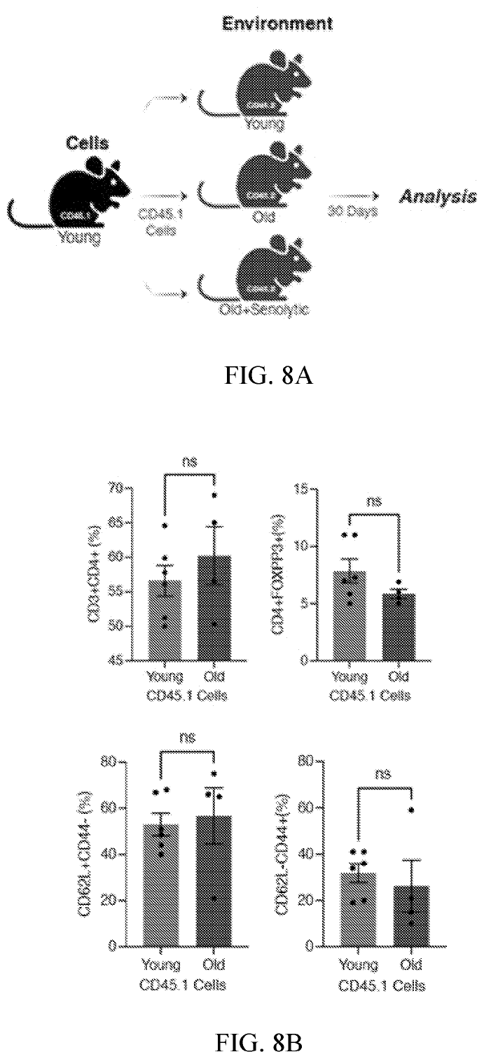

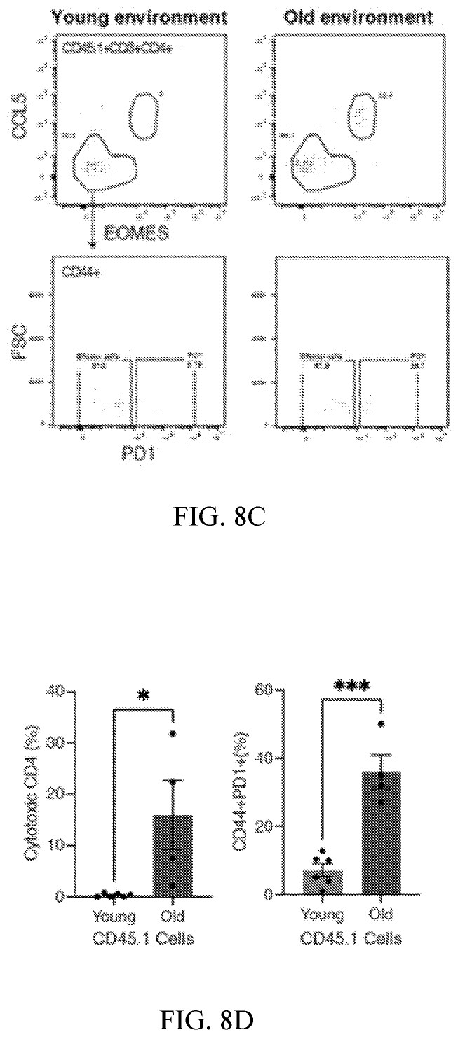

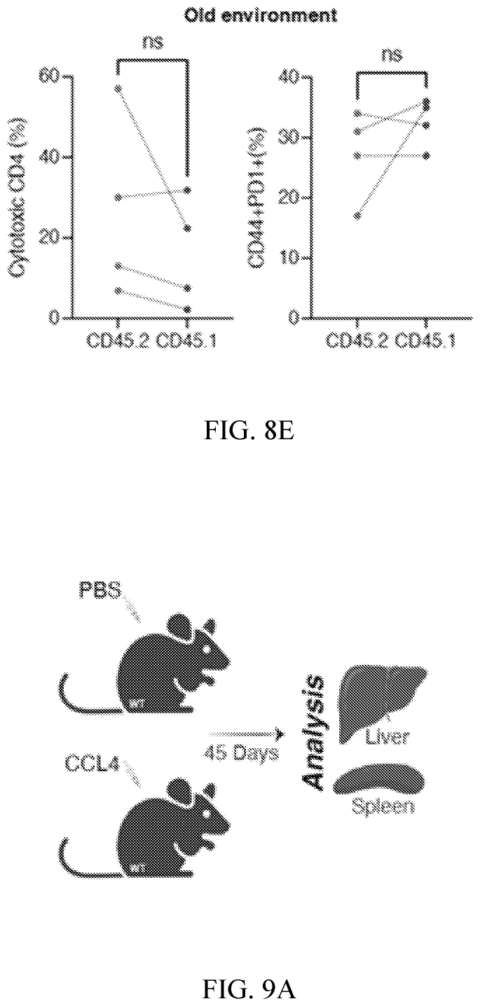

In order to evaluate the impact of ageing on T-cell population distribution, splenocytes obtained from young (1-month-old) CD45.1 mice were injected into two groups of mice: (a) young B6 WT mice (2 months old) and (b) old B6 WT mice (26 months old). Thirty days after the injection, the spleens were harvested and analyzed via flow cytometry for T-cell distribution, as outlined in FIG. 8A.

As seen from FIG. 8B, the percentage of CD4 cells (defined as CD45.1+, CD3+ and CD4+), CD4 Treg cells (defined as CD4+ and FOXP3+), effector memory cells (defined as CD44+ and CD62L- and naïve CD4 T cells (defined as CD62L+ and CD44— did not differ between the two groups.

However, as seen from the representative flow cytometry plots of FIG. 8C and FIG. 8D, gating cytotoxic CD4 T cells (defined as CD45.1+, CD3+, CD4+, EOMES+, CCL5+) and exhausted CD4 cells (defined as CD45.1+CD3+CD4+CD44+PD1+), significantly higher levels of these cell populations were found in splenocytes injected into an old environment, as compared to when injected into a young environment.

Importantly, a comparison between the percentage of endogenous CD4 T cells (CD45.2) and exogenous CD4 T cells (cd45.1) in the old mice showed no difference in the abundance of cytotoxic CD4 T cells and exhausted effector cells (CD44+PD1+) (FIG. 8E), confirming that CD4 T-cells are stimulated to become cytotoxic in an old spleen environment, irrespective of their origin (CD45.1 mice or B6 WT mice).

### Example 12—Evaluation T-Cell Subsets in a Mouse Model of Liver Fibrosis

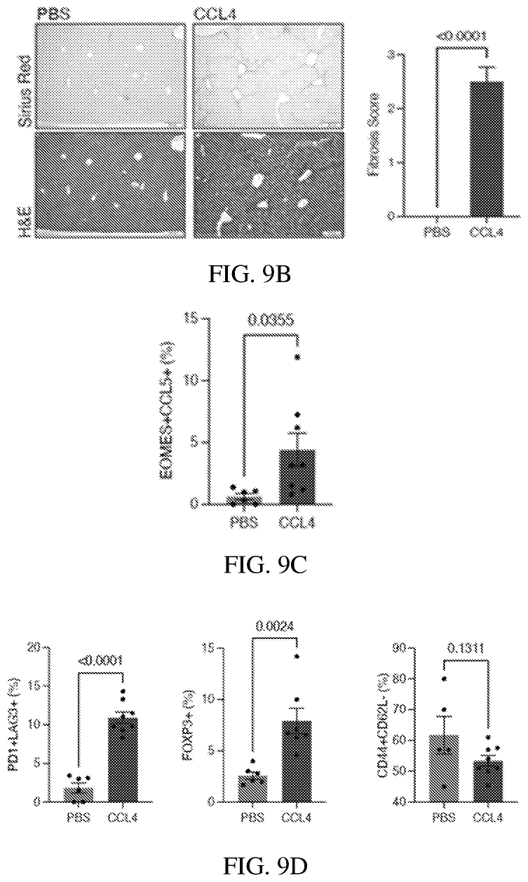

Carbon tetrachloride treatment induces liver senescence and liver fibrosis in mice. Accordingly, using this model, liver, spleen and blood may be analyzed for their T-cell distribution, as essentially outlined in FIG. 9A.

As seen from FIG. 9B, histological assessment of the livers (Hematoxylin & eosin staining and Sirus Red staining) showed a significant difference in the fibrosis score between Carbon tetrachloride treated and non-treated mice (P value 0.0001>).

Notably, as seen from the histograms of FIG. 9C (derived from flow cytometry analysis), the percentage of CD4-CTLs in the liver (defined as EOMES+ and CCL5+) out of the total CD4+CD8− cells, showed a significant increase in the CD4-CTL population after carbon tetrachloride injections (p-value=0.0355) indicating that CD4-CTLs play a role in cell senescence and as a result thereof, fibrosis.

Furthermore, as seen from the histograms of FIG. 9D (derived from flow cytometry analysis), the percentage of exhausted CD4 T-cells (defined as PD1+ and LAG3+) and Treg cells (defined as FOXP3+) were likewise higher in carbon tetrachloride treated mice as compared to non-treated mice. No significant difference in the effector memory cells (CD44+CD62L−) was found.

### Example 13—Evaluation T-Cell Subsets in a Mouse Model of Liver Fibrosis Using Eomes KO Mouse Model

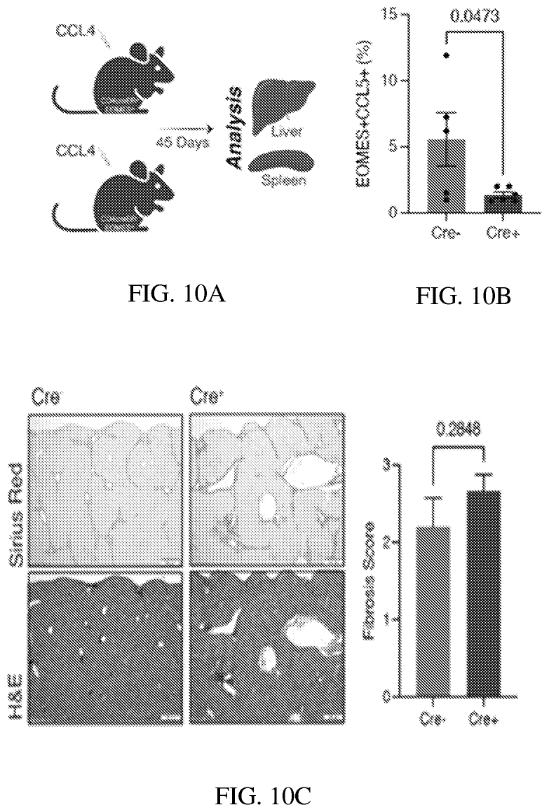

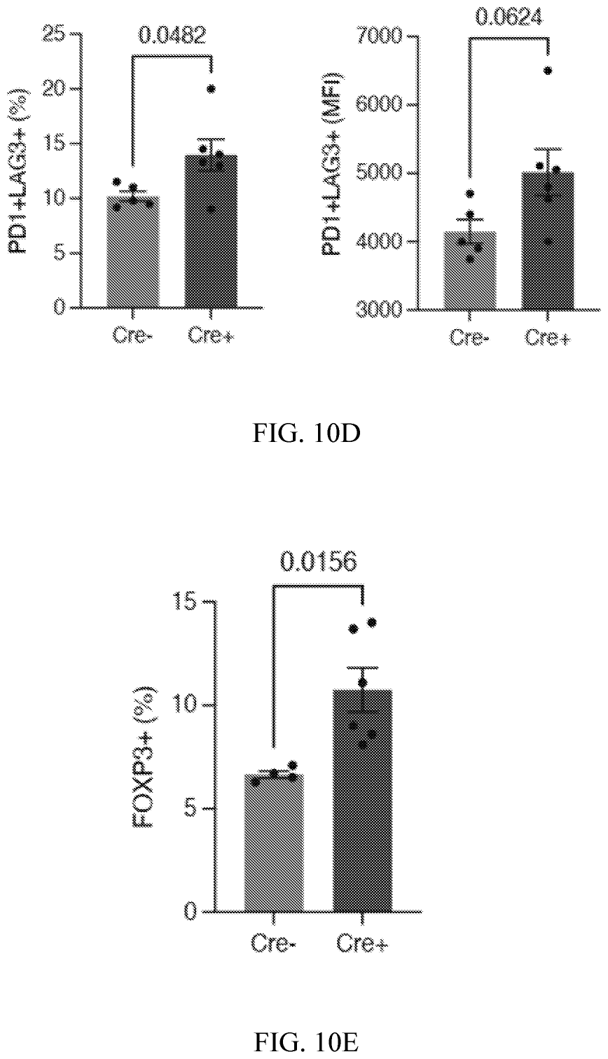

In order to further evaluate the role of CD4-CTLs in senescence, Eomes KO mice (CreER+) and control mice (CreER−) were injected with Tamoxifen prior to and during treatment with carbon tetrachloride. After 48 days, liver, spleen and blood were analyzed, as outlined in FIG. 10A.

As seen from FIG. 10B, the percentage of CD4-CTLs (EOMES+ and CCL5+) out of the total CD4+CD8− cell population was significantly higher in the WT mice (Cre−) as compared to the Eomes KO mice (Cre+) (p value=0.0473) indicating the efficiency of the tamoxifen injection causing KO of Eomes.

As seen from FIG. 10C, which shows representative histological stainings of the livers (Hematoxylin & eosin staining and Sirus Red staining), an increased fibrosis score was obtained in the Eomes KO mice, as compared to control mice (p-value=0.2848), indicating that without CD4-CTLs, cells undergo enhanced senescence causing increased fibrosis.

Moreover, as seen from FIG. 10D and FIG. 10E, the Eomes KO caused proliferation of exhausted CD4 cells as well as in Treg, in correspondence with the increased senescence and fibrosis.

### Example 14: Exploring Role of CD4-Populations in Tumors

In order to evaluate the role of CD4 T-cells in cancer, young (2 month) and old (12+ months) C57BL6 mice were injected with either cells3*10⁶ cells/injection obtained from an aggressive or less aggressive and more immunogenic head and neck squamous cell carcinoma tumors, as outlined in table 2.

TABLE 2

| | Young mice | Old mice |
|---|---|---|
| | (n = 23) | (n = 23) |
| High-grade tumor | 9 | 9 |
| Low-grade tumor | 9 | 9 |
| Control | 5 | 5 |

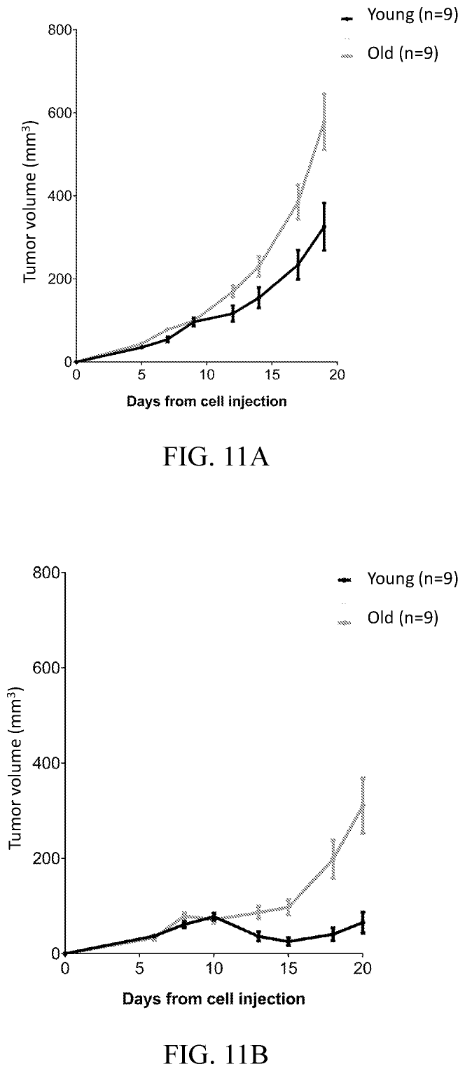

Tumor size was evaluated over time. As seen from FIG. 11A (high grade tumor) and FIG. 11B (low-grade, immunogenic tumor), in both old and young mice the tumor size obtained was significantly larger in the mice injected with the high-grade tumor cells as compared to mice injected with the low-grade tumor cells. Moreover, in both instances the tumors reached a significantly larger volume in the old mice as compared to young mice. The tumors and the spleens of the mice were harvested and evaluated for CD4 T-cell subset distribution by FACS using subset-specific markers, as essentially described herein.

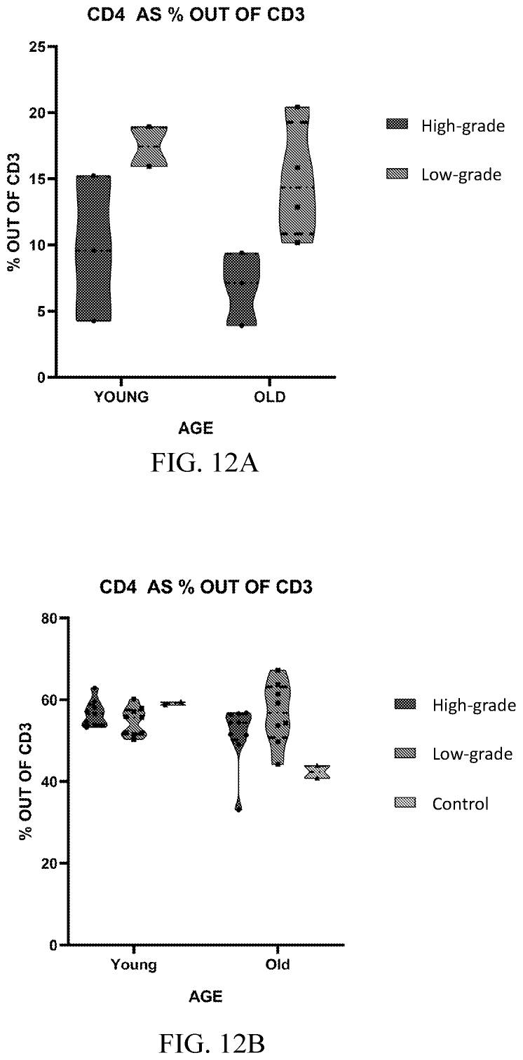

As seen from FIG. 12A which shows the percentage of CD4 T-cells out of the total CD3-positive population within the harvested tumor, in young mice the percentage of CD4 T-cells is significantly higher in the low-grade tumor as compared to the high-grade tumor, as expected. While a same trend was observed in the old mice, the percentage of CD4-T-cells was lower than that observed in the young mice in the high-grade tumor, and in the low-grade tumor a much higher variability in the level of CD4 T-cells was observed. Moreover, as seen from FIG. 12B which shows the percentage of CD4 T-cells out of the total CD3-positive population in the spleen, in both groups, the percentage of CD4 T-cells was higher than that of the control.

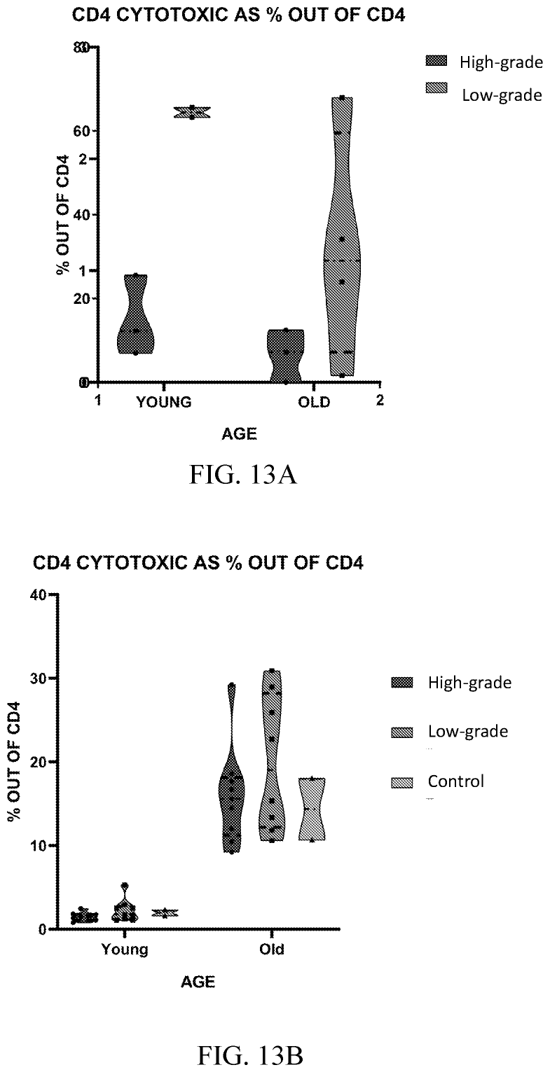

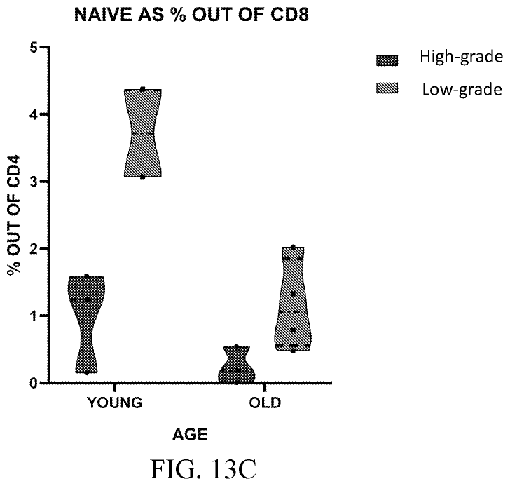

When specifically evaluating the level of CD4 CTLs, an increase in the percentage of CD4 CTLs in the spleen was observed in all old mice as compared to young mice (see FIG. 13B). However, in the tumors of the young mice, the level of CD4 CTLs was significantly higher in the low-grade tumors as compared to the high-grade tumor (see FIG. 13A), indicating that CD4 CTLs are involved in the immunogenicity of the low-grade tumor. Importantly, in the old mice, the percentage of CD4 CTLs was lower in both tumors indicating that the increase in tumor size observed in the older mice may be at least partially due to an attenuated CD4 CTL response. In the low-grade tumor, a large variability in the percentage of CD4 T-cells in the grade tumor was observed, which may go hand in hand with the variability observed in the tumor size in this group of mice (old—injected with low-grade tumor cells)—data no shown.

Notably, a similar pattern was observed for the naïve CD-4 T-cells, which were found at high-levels in the low-grade tumor, but not in the high-grade tumor of the young mice (FIG. 13C), indicating the importance of the naïve-cells in attenuating tumor aggressiveness. In the old, mice a higher level was also found in the low-grade as compared to high-grade tumor (FIG. 13C). However, in general a much lower level of naïve cells was found in the old mice as compared to the old mice, indicating the role of the naïve-T-cells in the correlation between age and tumor aggressiveness.

### Example 15: Exploring the Impact of the CD4-CTLs on Tumors in CD4-CTL Depleted Mice

The role of CD4-CTLs in tumors is assessed in CD4CreER⁻ EOMES^{lox/lox} mice and littermate control mice induced to form a tumor (e.g. by orthotopic injection of B16 melanoma cells). Tumor size and/or tumor progression is evaluated e.g., by imagining.

### Example 16: Exploring the Impact of the Cytotoxic CD4 T-Cell Subset on Alzheimer

The role of CD4-CTLs in Alzheimer's disease is assessed in a mouse model of Alzheimer's disease (AD)-like pathology (Eremenko, 2019 Mittal, 2019; Strominger, 2018). Bone marrow chimera mice are generated by transplanting the bone marrow of CD4CreER-EOMES^{lox/lox} mice into the 5XFAD mouse model of AD at 6-8 months of age. Two months later, the amyloid deposition and the associated pathology in the brain, are analyzed as essentially described (Eremenko E. et al., EBioMedicine. 2019 May; 43:424-434and Mittal K. et al., iScience. 2019 Jun. 28; 16:298-311).

### Example 17: Treating Alzheimer by Administration of aTregs

aTreg cells are injected IP or IV into 5XFAD chimera mice at age 10-12 mo. Two months later, the amyloid deposition and the associated pathology in the brain, are analyzed as essentially described (Eremenko, ibid. and Mittal, ibid.).

### Example 18: Treating Alzheimer by Depletion of CD4-CTLs

Bone marrow of CD4CreER-EOMES^{lox/lox} is transplanted into 5XFAD mice. Two months later, the amyloid deposition and the associated pathology in the brain, are analyzed as essentially described (Eremenko, ibid. and Mittal, ibid.).

Alternatively, CD4-CTLs are depleted by weekly IP injections of anti-IL27 (25 microgram/mouse) into wt C57BL6 mice aged 18-20 months. 1-2 months later, the amyloid deposition and the associated pathology in the brain, are analyzed as essentially described (Eremenko, ibid. and Mittal, ibid.).

### Example 19: Treating Tumors by Administration of CD4-CTLs

CD4-CTLs are injected IV or IP into control and/or CD4^{CreER}-EOMES^{lox/lox} mice induced for tumor formation. Tumor size and/or tumor progression is evaluated, e.g. by imagining.

### Example 20: Differentiating CD4 T-Cells into CD4-CTLs

Fibroblasts are induced by gamma-radiation to become senescent. Subsequently the fibroblasts are incubated with effector memory cells with or without inflammatory cytokines such as IL-1, TNF, IL-6, TGFb, IFN-g alone or in combination for 1-6 days. CD4-CTL differentiation and proliferation is inspected by flow cytometry. Cytokines are validated by neutralizing ab's to specific cytokines. In order to evaluate whether the differentiation depends on antigen presentation the cells are co-cultured with MHCII blocking ab's.

Alternatively, CD4-CTLs are generated by retroviral transduction to overexpress the key transcription factors of CD4-CTLs, specifically EOMRS, Runx3, Tbet, RORa. Effector memory CD4 T cells are isolated and undergo activation while being transduced with retroviral vectors to over express one or more of the transcription factors.

1-37. (canceled)

38. A method for treating a senescence-associated or an immune-inflammatory associated disease in a subject in need thereof, the method comprising:
a) obtaining information of a disease etiology of the subject; wherein the disease etiology comprises a senescence-associated or an immune-inflammatory associated disease; and
b) administering to the subject cytotoxic CD4 T-cells (CD4-CTLs) and/or an agent capable of inducing CD4-CTLs differentiation and/or proliferation, thereby treating the senescence-associated disease; or administering an agent capable of inducing aTreg depletion and/or inhibition.

39. The method of claim 38, wherein the cytotoxic CD4 T-cells are autologous to the subject.

40. The method of claim 39, wherein the method further comprises a step of isolating effector memory CD4 T-cells (EMs) from the subject and cause their differentiation into CD4-CTLs; wherein causing their differentiation comprises cultivating the EMs in the presence of one or more marker selected from IL-27, IL-6, IL1, TNF; and wherein isolating EMs comprises sorting EMs from the subject using CD44, CD62L, CD45, Itgb7 and/or IL-18R1 as biomarkers.

41. The method of claim 38, wherein the method further comprises a step of isolating and proliferating aTregs CD4 T cells isolated from the subject; wherein the isolating comprises sorting aTregs from the subject using one or more biomarkers selected from CD137, CD134, FOXP3+, GITR+, Helios+, CD74, HLA-DR CD81, TIGIT, PD1.

42. The method of claim 38, wherein the immune-inflammatory associated disease etiology is an autoinflammatory and/or autoimmune disease and wherein the cell-based therapy comprises administering to the subject aTregs CD4 T cells and/or an agent capable of inducing CD4 aTregs differentiation and/or proliferation.

43. A method for restoring and/or adjusting an immune system of a subject, the method comprising:
c) obtaining a biological sample from a subject, the biological sample comprising one or more subsets of CD4 T-cells;
d) identifying the presence, frequency and/or ratio of cytotoxic CD4 T-cells (CD4-CTLs), exhausted CD4 T-cells and/or aTreg cells, and
e) providing cell-based therapy to the subject based on the identification, thereby restoring and/or adjusting the immune system of the subject.

44. The method of claim 43, wherein said identifying the presence of one or more subsets of CD4 T-cells comprises determining the amount of each identified subset of CD4 T-cells relative to a control value.

45. The method of claim 43, further comprising identifying the presence, frequency and/or ratio of one or more subsets of CD4 T cells in the immune system, selected from activated regulatory effector memory CD4 T-cells (EMs); naïve CD4 T-cells, naïve_Isg15 CD4 T-cells, rTregs CD4 T-cells or any combination thereof.

46. The method of claim 43, wherein the identifying is based on the level of one or more biomarkers associated with the CD4-CTLs; and wherein the identifying is based on a plurality of biomarkers selected from Nkg7, Runx3, EOMES, Gzmk, IFN-b, IFN-g, IL-27, IL21, IL 17A, Ccl3, Ccl4 and Ccl5.

47. The method of claim 46, wherein the biomarker is EOMES.

48. The method of claim 43, wherein the therapy comprises administering to the subject CD4 aTregs and/or an agent capable of inducing CD4 aTregs differentiation and/or proliferation.

49. The method of claim 48, wherein the aTregs CD4 T cells are autologous to the subject.

50. The method of claim 49, wherein the method further comprises a step of isolating and proliferating aTregs CD4 T cells of the subject; and wherein the isolating comprises sorting aTregs from the subject using one or more biomarkers selected from CD137, CD134, FOXP3+, GITR+, Helios+, CD74, HLA-DR, CD81, TIGIT, PD1.

51. The method of claim 43, wherein the therapy comprises administering to the subject an agent targeting the CD4-CTLs; wherein the agent is selected from the group consisting of an antibody, a siRNA, a microRNA, a small molecule or any combination thereof.

52. The method of claim 51, wherein the antibody is an NKG2D antibody, a CD7 antibody, a CD134 antibody, a CD137 antibody, a GITR antibody, a CCL5 antibody, an IL-27 antibody or any combination thereof.

53. The method of claim 43, wherein the therapy comprises administering to the subject CD4-CTLs and/or an agent capable of inducing CD4-CTL differentiation and/or proliferation; wherein the CD4-CTLs are autologous to the subject.

54. The method of claim 53, wherein the method further comprises a step of isolating effector memory CD4 T-cells (EMs) from the subject and cause their differentiation into CD4-CTLs; wherein isolating EMs comprises sorting EMs from the subject using CD44, CD62L, CD45, Itgb7 and/or IL-18R1 as biomarkers.

55. The method of claim 43, further comprising evaluating a grade of tissue senescence based on the presence, frequency and/or ratio of cytotoxic CD4 T-cells (CD4-CTLs), exhausted CD4 T-cells, aTreg cells or combinations thereof.

56. A pharmaceutical composition for treating an immune system imbalance, the composition comprising isolated CD4 T-cells and one or more excipients; wherein the immune system imbalance is related to a senescence-associated disease and wherein the CD4 T-cells are CD4 CTLs; and/or wherein the immune system imbalance is related to an autoinflammatory or autoimmune disease and wherein the CD4 T cells are CD4 aTregs.

57. The pharmaceutical composition of claim 56, wherein the senescence-associated disease is selected from frailty, cancer, chronic infection, chronic inflammation, Alzheimer's disease, dementia, Parkinson's disease, tissue senescence or any combination thereof.
